(19) United States
(12) Patent Application Publication
(10) Pub. No.: US 2013/0089599 A1
(43) Pub. Date: Apr. 11, 2013

(54) NANO-ENCAPSULATED THERAPEUTICS FOR CONTROLLED TREATMENT OF INFECTION AND OTHER DISEASES

(71) Applicants: Mauris N. DeSilva, Austin, TX (US); Karen O'Connor, Scoresby (AU); Amer Tiba, Chicago, IL (US)

(72) Inventors: Mauris N. DeSilva, Austin, TX (US); Karen O'Connor, Scoresby (AU); Amer Tiba, Chicago, IL (US); Patty-Fu Giles, University Park, IL (US)

(73) Assignee: The United States of America as Represented by the Secretary of the Navy, Arlington, VA (US)

(21) Appl. No.: 13/492,889

(22) Filed: Jun. 10, 2012

Related U.S. Application Data
(60) Provisional application No. 61/495,909, filed on Jun. 10, 2011.

(51) Int. Cl. A61K 9/51

(52) U.S. Cl. CPC A61K 9/51; Y10S 977/773; B82Y 5/00
USPC 424/450; 424/490; 424/493; 977/773; 977/906; 977/907

(57) ABSTRACT

This invention relates to a method to provide immediate, direct and controlled time release of an effective amount of therapeutics to a wound site for a prolonged period. The pharmaceutical formulation comprising a plurality of nanoparticles, said nanoparticles encapsulating a therapeutically effective amount of one or more antibacterial agents, and an application of the formulation to an implant before surgery provide for extended release of said antibacterial agents.

### CROSS-REFERENCE TO RELATED APPLICATION

This application claims priority to U.S. provisional application 61/495,909 filed Jun. 10, 2011 and PCT application PCT/US2011/042776 filed Jul. 1, 2011, which is hereby incorporated by reference in its entirety.

### FIELD OF INVENTION

This invention relates to a pharmaceutical formulation for extended release of antibiotics using nanoparticles. The invention also relates to a method of releasing antibiotics directly to a surgical site for an extended period to treat infections.

### BACKGROUND

Blast-injured warfighter often suffers severe trauma to their body. It is of importance for the military to reduce patient recovery time, and minimize potential pose-surgical complications. However, bacterial infection due to multi-drug resistance bacteria is a current medical challenge in traumatic injury treatments. These infections can delay wound healing, and increase the rate of mortality in severe cases.

For example, blast-injured warfighter often suffers head and facial trauma. At the Walter Reed Army Medical Center, National Naval Medical Center, and Naval Postgraduate Dental School, cranial plate implantation has been a necessary, and accepted treatment for many types of blast injuries to the head. However, severe bacterial infection of the soft tissue surrounding the brain is frequently observed in these patients, which causes additional complication to treatment. Some patients also develop other infections after receiving craniofacial implants. As a result, the patients often require additional invasive surgical procedures to remove the infection. There is a need to effectively prevent and control post-surgical infections.

Imipenem, Tobramycin, Clindamycin, Vancomycin and Rifampicin are the primary antibiotics used to treat infections in implant patients. These antibiotics are usually administrated orally, absorbed from the gastrointestinal tract, extensively metabolized in the liver, and then distributed throughout the body. A small amount of antibiotics adequate therapeutic concentration of the drug (approximately 5-10%) will reach the surgical site in approximately 1.5 to 5 hours after the administration. Hence, there is a need to have a method of delivering immediate, direct, and continuous administration of antibiotics at the surgical site to prevent and control post-surgical infections. A targeted drug delivery system can help reduce dangerous side effects of systemic high-dose antibiotic treatment. It can also eliminate the time that otherwise is required for the drugs to be processed by the liver while providing improved antimicrobial efficacy against drug-resistant bacterial strains (Nandi I, 2003; Torchilin V P., 2001). Local administration of encapsulated antibiotic offers such a solution. The method allows antibiotic agents to be directly administered at the targeted site, which provides controlled and continuous release of the antibiotics against a broad spectrum of bacteria, over a prolonged period with minimum side effects.

The majority of cranial implants are made from polymethylmethacrylate (PMMA), a synthetic, biocompatible polymer resin, which has been used in medical applications since 1933 (Boger A, 23 Aug. 2007; and Frankel B M, 2007). PMMA has a good degree of compatibility with human tissue, and have been approved to for use as bone cement, replacement intraocular lenses, and denture materials. PMMA cranial implants may be formed intraoperatively from cured solid compositions or preoperatively fabricated using information from patient CT scans in combination with stereolythography. PMMA embedded antibiotics have been used for the prevention of post-surgical infections (Mohanty et al., 2003). However, until now there is no study on incorporating encapsulated antibiotics onto the implant to provide controlled and continuous delivery of a drug. Previous use of PMMA for antibiotic delivery involves multiple replacements of the PMMA beads. The beads were originally placed adjacent to a surgery site (e.g. knee), which were later removed from the surgery region. However, this method is not possible with cranial implants. There's not excess room around cranial implant site to place PMMA beads like there is in knee surgery. In addition, removals of PMMA beads require performance of additional surgical procedures and may have post surgical complications.

Nanotechnology has been applied to solving the problems associated with traditional delivery systems, and can be used for targeted and controlled delivery (Alipour, 2010; Lanio M E, 2008). The majority of nanoparticulate drug delivery system has focused on using nanoparticles as polymeric carriers for anticancer agents or in gene delivery and tissue engineering (Henry et al, 2002; Pridgen et al, 2007). Nanoparticles such as liposome and micelles have been used in the past to protect drugs and prolong drug release by isolating them from systematic degrading enzymes, and promoting their diffusion across the bacterial envelope (Torchilin, 2001; Muller-Goymann, 2004). It has been shown that nanoparticles encapsulated drug delivery systems can improve antimicrobial efficacy against drug-resistant strains (Torchilin, 2001; Nandi et al., 2003). However, this nanoparticle delivery system has not been extended to use in implants. There are no reports on studies using liposome/micelles encapsulated antibiotics to prevent and treat post-surgical infections.

### SUMMARY OF INVENTION

Accordingly, an object of this invention is a pharmaceutical formulation comprises a combination of different nanoparticles having different sizes and properties, each encapsulating therapeutically effective amount of one or more antibacterial agents. The nanoparticles may be incorporated onto an implant.

Another object of the invention is a method to provide immediate, direct, and continuous administration of an effective amount of one or more therapeutic agent at a target site in a controlled-released manner for an extended period.

A still further object of the invention is a method to provide immediate, direct, and continuous administration of a therapeutic agent at a target site to prevent and treat infection.

In a preferred embodiment, a combination of nanoparticles of different types and sizes are utilized based on a prescribed antibiotic treatment regimen for a patient. These nanoparticles are incorporated onto the surface of a PMMA or titanium implant. The nanoparticles encapsulate an effective amount of at least one type therapeutic agent, such as an antibiotic agent. Once in place, the nanoparticles administer direct, immediate, and continuous treatment to a site in a controlled-release fashion for an extended period. This pharmaceutical formulation may also be used to administer other molecules such as anti-cancer treatment, pain medication or growth hormone. It may also be used in other surgical procedures to combat post-operation infection, including but not limited to other bone replacement and joint or hip surgery.

### DESCRIPTION OF FIGURES

FIG. 1: Antibacterial activities of Tobramycin/Rifampicin cocktail.

FIG. 2. Effect of Temperature on Antibiotic Functionality.

FIG. 3A mount of antibiotics retained in liposomes after 9 days.

FIG. 4. Comparison of free Tobramycin and Tobramycin Encapsulated Liposomes against S.a and S.a-R.

FIG. 4: Comparison of Single Antibiotic Liposomes and Cocktail of Antibiotic Liposomes.

FIG. 5 Antibacertial efficacy of Antibiotic Liposomes

FIG. 6 Antibacterial efficacy of Antibiotic Liposomes Coating on Titanium Implant

FIG. 7 Antibacterial efficacy of Antibiotic Liposome Coating on PMMA implant

FIG. 8 The effect of PVA concentration on the particle sizes (a) and encapsulation efficiency of rifampicin-loaded nanoparticles (b).

FIG. 9. Influence of water phase volume on the particle sizes with PLGA 502H and 504 as polymers.

FIG. 10. In vitro release of rifampicin and tobramycin from loaded nanoparticles.

### DETAILED DESCRIPTION

A pharmaceutical formulation of this invention comprising a plurality of nanoparticles, said nanoparticles encapsulating a therapeutically effective amount of one or more therapeutic agents, and an application of the formulation to an implant before surgery provide for extended release of the therapeutic agents. The therapeutic agent encapsulated may be an antibiotic, such as silver ion. Other therapeutic compound may also be delivered, including but not limited to an anti-cancer drug, pain medication, or a growth hormone. The antibiotic may be used in this pharmaceutical formulation may be selected from the group consisting of Imipenem, rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof. The pharmaceutical formulation may be coated on the surface of an implant using a physiological acceptable coating material to stabilize said nanoparticles, such as a modified PMMA compound or Chitosan, or by phage display. Implants may be coated by this pharmaceutical formulation include but not limited to PMMA implant, hydroxyapatite implant, hydrogel or titanium implant.

The pharmaceutical formulation of the instant application has distinct major advantages over the current delivery system for antibiotic treatment of infection. Using the current systemic delivery method, only 5-10% of antibiotics is delivered to the required area (Giorgio et al, 1998). By using a drug delivery system, such as the inventive pharmaceutical formulation, which specifically targeting the infection area, unnecessary delivery to other part of the body are greatly reduced, avoiding dangerous side effects or overdose. The inventive pharmaceutical formulation also eliminates time otherwise needed for drugs to be processed by the liver, allowing immediate effective treatment of the area. As a result, fewer therapeutic agent need to be administered to a patient, offering immediate treatment at the site with lower risk.

The delivery system using this pharmaceutical formulation also has distinct advantage over current PMMA antibiotic drug delivery system, which involves direct embedment of antibiotics into the PMMA without nanoparticle encapsulation. Direct embedding antibiotic into PMMA beads hinders antibiotic release, and requires multiple replacements of PMMA beads, which exposes the tissues to more injuries, and subject the patient to potential secondary infections.

Furthermore, the delivery system using the inventive pharmaceutical formulation may be customized according to the needs of each patient. This is accomplished by varying the entrapped antibiotics and their concentrations. Different nanoparticles can be used in one pharmaceutical formulation depending on the therapeutic agents prescribed. A combination of different type of nanoparticles in a pharmaceutical formulation can also provide controlled release of drug, and the desired efficacy. Most importantly, the nanoparticles used in this drug delivery system are composed of biomaterials that are already proven safe to be used in many FDA approved drug delivery systems.

Prolonged and controlled release of therapeutics depends on the properties and sizes of nanoparticles used. A single type or a combination of different types of nanoparticles may be used for the drug delivery of the present invention, including but not limited to micelles, inverse micelles, liposomes and a variety of known polymeric nanoparticles. Each type of nanoparticle having a different half-life for drug release and a different particle size.

Typical micelles have a hydrophobic core and a hydrophilic surface allowing the encapsulation of hydrophobic molecules in an aqueous solution. Inverse (or reverse) micelles, with a hydrophilic core, can be produced via microemulsion method. In microemulsions, two immiscible phases (water and ‘oil’) are present with a surfactant, the surfactant molecules may form a monolayer at the interface between the oil and water, with the hydrophobic tails of the surfactant molecules dissolved in the oil phase and the hydrophilic head groups in the aqueous phase. As in the binary systems (water/surfactant or oil/surfactant), self-assembled structures of different types can be formed, ranging, for example, from (inverted) spherical and cylindrical micelles to lamellar phases and bicontinuous microemulsions, which may coexist with predominantly oil or aqueous phases. This type of micelle is specifically useful in encapsulating hydrophilic molecules.

Liposomes are colloidal lipid bilayer vesicles ranging from a few nanometers to several micrometers in diameter. They can safely entrap hydrophilic molecules in the core, and hydrophobic molecules in the lipid bilayer in an aqueous solution. Liposomes can be composed of naturally-derived phospholipids with mixed lipid chains (like egg phosphatidylethanolamine), or of pure surfactant components like DOPE (dioleoylphosphatidylethanolamine. The original liposome preparation of Bangham et al. (J. Mol. Biol., 1965, 13:238-252) involves suspending phospholipids in an organic solvent which is then evaporated to dryness leaving a phospholipid film on the reaction vessel. An appropriate amount of aqueous phase is then added, the mixture is allowed to “swell,” and the resulting liposomes which comprise multilamellar vesicles (MLVs) are dispersed by mechanical means. This technique provides the basis for the development of the small sonicated unilamellar vesicles described by Papahadjopoulos et al. (Biochem. Biophys. Acta., 1967, 135:624-638), and large unilamellar vesicles. Unilamellar liposomes may be synthesized by reverse-phase evaporation technique, while multilamellar liposome vesicles will be formulated using the lipid hydration technique (Mugabe et al., 2006a, Mugabe et al., 2006b). A review of methods for producing liposome, micelles and inverse micelles is provided in Liposomes, Marc Ostro, ed., Marcel Dekker, Inc. New York, 1983, the relevant portions of which are incorporated herein by reference. See also Szoka, Jr. et al., (Ann. Rev. Biophys. Bioeng., 1980, 9:467), the relevant portions of which are also incorporated herein by reference.

Liposomes that contain low (or high) pH can be constructed such that dissolved aqueous drugs will be charged in solution (i.e., the pH is outside the drug's pH range). As the pH naturally neutralizes within the liposome (protons can pass through some membranes), the drug will also be neutralized, allowing it to freely pass through a membrane. These liposomes work to deliver drug by diffusion rather than by direct cell fusion. Another strategy for liposome drug delivery is to target endocytosis events. Liposomes can be made in a particular size range that makes them viable targets for natural macrophage phagocytosis. These liposomes may be digested while in the macrophage's phagosome, thus releasing its drug. Liposomes can also be decorated with opsonins and ligands to activate endocytosis in other cell types.

Polymeric nanoparticles may be prepared using several polymers. Polycaprolactone, poly(alkyl cyanoacrylates), and poly (lactic-co-glycolic acid) were commonly used. Among them, the best known class of the polymers for drug delivery is poly (dl-lactic-co-glycolic acid) (PGLA) which is biodegradable and biocompatible. While PLGA nanoparticles have been extensively studied in various aspects including anti-cancer studies, their role in antibiotic delivery remains a relatively under investigated field.

Several methods for the polymeric nanoparticle production have been developed which include emulsification solvent evaporation, emulsification solvent diffusion, emulsification reverse salting-out, and nanoprecipitation. These methods generally include two main steps: to prepare an emulsified system and to form nanoparticles. To encapsulate lipophilic and hydrophilic reagents, two types of preparation methods were commonly used. Oil in water (O/W) emulsification was used to load lipophilic drugs. Water in oil in water (W/O/W) double emulsification was used to load hydrophilic drugs. In general, nanoparticle and polymer molecule sizes are critical for the efficacy of the therapeutic agent in terms of tissue penetration, cellular uptake, release profile, and degradation behavior. In addition, poly(vinylalcohol) (PVA) plays an important role in stabilization of emulsification in the formation of NPs.

In an embodiment of the present invention, the controlled and prolonged release of therapeutic agents may be accomplished by manipulating the type and sizes of the nanoparticles of different properties. The drug delivery system of the present invention may comprise of a combination of unilamellar and multilamellar liposomes or micelles entrapping antibiotic agents. Having both types of liposomes allow for better control of release rate. For example, clindamycin is the drug of choice for treatment of infections of the brain. Clindamycin is hydrophilic, and therefore may be encapsulated in inverse micelles and liposomes. Inverse micelles are generally smaller, tighter, and more stable than liposomes. Therefore, by manipulating the concentrations, and sizes of liposomes and inverse micelles, the controlled release of encapsulated antibiotics over time may be achieved. The different release times of nanoparticles allows for sustained delivery of antibiotic agent over time. Similarly, a combination of inverse micelles and lipsosomes can be used for the encapsulation of any hydrophilic drugs such as Imipenem, vancomycin, gentamicin, gentamicin sulfate, tobramycin, ampicillin, penicillin, ethambutol, clindamycin, and a cephalosporin including cefazolin, ceftriaxone and cefotaxime for bacterial infections, acyclovir for viral infections, and amphotericin B for fungal infections. For delivery of a hydrophobic drug, micelles may be used.

The nanoparticle drug delivery system of the present invention may be customized according to the needs of each patient by varying therapeutic agents entrapped, and the mixture of nanoparticles used according to the prescription. In an embodiment of the inventive method to treat infection, a single or a combination of therapeutic agents may be used, including but not limited to silver ion, Imipenem, rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime. Antibiotic cocktails provide better efficacy against a wider range of infection. Other therapeutic agents such as pain medication may also be included in the formulation to relieve pain.

Liposomes and micelles are completely biodegradable and non-toxic. Drug delivery systems using these nanoparticles have been extensively studies for their ability of delivering therapeutic drugs since year 2000 (Arkadiusz et al., 2000). Nanovesicles/nanoparticles used in an embodiment of the drug delivery system of this invention are composed of organic materials, which are already used in many FDA approved drug delivery systems such as AMBISOME™ (Astellas Pharma US, Inc).

In case of an implant, in order to deliver antibiotic agents directly to the site, the nanoparticles may be applied to the implant, allowing sustained, localized release of the antibiotics. Nanoparticles may be simply coated on to the surface of an implant and allowed to dry pre-operation. To stabilize the nanoparticles coating, a stabilizer such as Chitsan may be added.

Nanoparticles may be embedded onto the surface of an implant via a secondary coating of acrylate/methacrylate polymer resin, which is chemically altered PMMA, and can set quickly using photo-initiated polymerization (light curing) or autopolymerization (chemical curing). PMMA is an excellent material for seeding and coating nanoparticle-encapsulated antibiotic with its high surface area and low density. However, because different PMMA compounds works differently with each hydrophobic or hydrophilic molecules, PMMA coatings need to be tested for compatibility with the therapeutic agent of interest.

In an alternative embodiment, nanoparticles may be attached to the PMMA implant via phage display. Phage display is a powerful tool for binding proteins to non-proteinaceous materials (Whaley et al. 2000, Faduka et al., 2006). This method has been used for antibodies, receptors, semi-conductors, and organ targeting (Arap et al., 2002; Flint et al., 2005; Johanson et al., 2005; O'Connor et al., 2005; O'Connor et al., 2006; Valadon et al., 2006). In vitro phage display may be utilized to select a specific anchoring peptide, which binds directly to PMMA. Phage displayed random peptide libraries (˜1⁰¹⁰ transducing units) are exposed to PMMA beads. Following multiple rounds of selection, PMMA-specific phages will be harvested and the peptide-coding inserts will be sequenced. Secondary structure motifs of selected peptides will be assessed by computer simulator and PMMA binding will be determined through microscopy and ELISA. PMMA-binding peptides will be incorporated into the surface of the nanoparticles to promote strong attachment of the nanoparticles to the PMMA implant.

In yet another embodiment, in vivo phage display may be used to identify peptides, which target the brain tissue. Random phage libraries will be injected intravenously into mice. Phages that successfully penetrate the blood brain barrier will be harvested and the peptide-coding inserts will be screened and sequenced. Secondary structure motifs of selected peptides will be assessed to determine if they will carry nanomicelles through the blood brain barrier. Selected peptides will be incorporated onto nanomicelles and injected into mice. The peptides will carry nanoparticles across the blood-brain barrier for delivery of antibiotics to the brain. The third approach may also be useful for treatment of bacterial encephalitis not resulting from surgery.

Nanoparticles encapsulating antibiotic agents may also be formulated as wound dressing, infection preventing gel or cream, and infection treatment such as photodynamic therapy.

Micelles may be used to incorporate various topical antibiotics, by embedding into semi-occlusive hydrogel wound dressings. Hydrogel dressing helps to create a moist wound environment, which facilitates drug delivery. The dressing also provides a soft, cushioning, and soothing cover over bony prominences or abraded skin. It will be easy to apply and remove by corpsmen in the field. The wounded soldiers will receive instant pain relief as well as needed protection of the wound against infections.

### Example 1

### Selection of Antibacterial Agents

In vitro antibacterial efficacy of five commonly-used antibiotics (Imipenem, Tobramycin, Clindamycin, Vancomycin and Rifampicin) was investigated against 4 bacterial strains (A. bumannii, P. aeruginosa, P. mirabilis and S. aureus). The amount of antibiotic required for 50% inhibition (MIC₅₀) is recorded in Table 1.

TABLE 1
in vitro antibacterial activity of individual antibiotic (mg/ml).

| Antibiotics | A. bumannii | P. aeruginosa | P. mirabilis | S. aureus |
|---|---|---|---|---|
| Imipenem | 10.25 ± 2.33 | 0.91 ± 0.56 | 1.02 ± 0.01 | 0.02 ± 0.02 |
| Tobramycin | 1.05 ± 0.02 | 0.44 ± 0.12 | 7.2 ± 0.73 | 0.43 ± 0.1 |
| Clindamycin | 2.72 ± 0.68 | >1000 | 23.88 ± 8.43 | 0.004 ± 0.002 |
| Vancomycin | 79.4 ± 4.1 | >1000 | >1000 | 0.82 ± 0.57 |
| Rifampicin | 0.085 ± 0.01 | 19.08 ± 6.14 | 3.77 ± 0.92 | 0.004 ± 0.002 |

Tobramycin, rifampicin and imipenem demonstrated better anti-bacterial activities against all 4 bacterial strains with MIC50 less than 10 μg/ml. In particular, two antibiotics, tobramycin and rifampicin showed the strongest activities to A. bumannii and S. aureus, in which MIC50s were equal or less than 1 μg/ml.

To evaluate antibacterial efficacy of an antibiotic cocktail vs. individual antibiotic, tobramycin and rifampicin were combined based on the amount of each antibiotic required for bacteria inhibition (MIC50) from Table 1, to form a cocktail. The antibiotics were combined at four times their specific MIC50's for each bacterium, and then serially diluted and inoculated onto four types of bacteria as before to determine the MIC50.

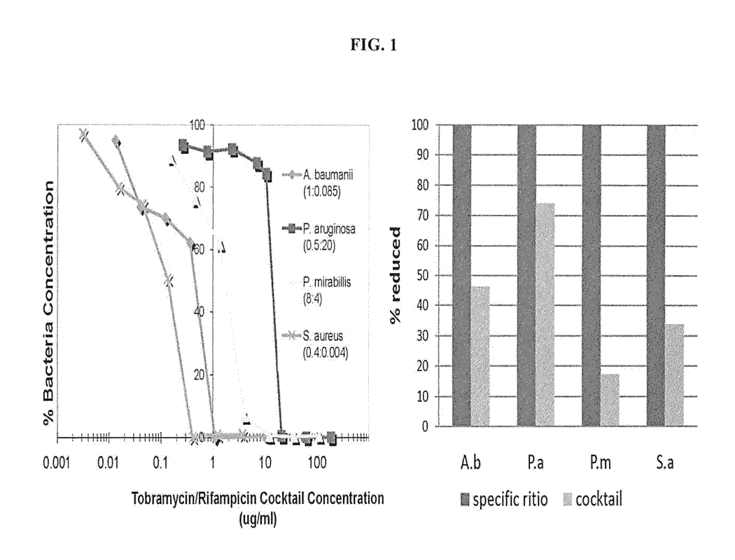

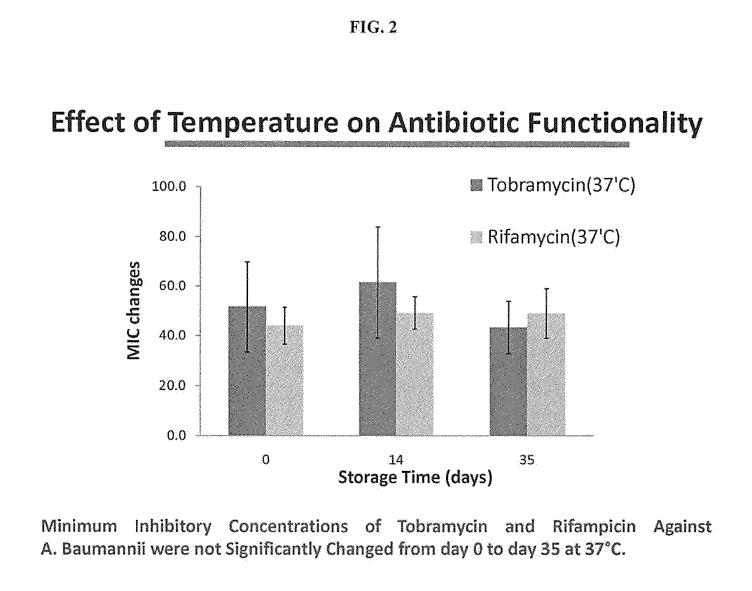

Results (FIG. 1) show that using tobramycin/rifampicin cocktail, MIC50 were decreased by 46%, 74%, 17% and 34% for A. baumannii, P. aeruginosa, P. miabilis and S. aureus, respectively. This suggests that less amount of antibiotics may be used to achieve the same efficacy of individual antibiotic.

Silver is a well-known, effective broad-spectrum antimicrobial agent. Silver ion solutions were added to the antibiotic cocktail solutions to determine whether silver ions can enhance antimicrobial activities of the cocktail and decrease amount of antibiotics used for MIC₅₀. Specifically, silver concentrations at an estimated MIC_{50, 25, 12.5}, were mixed with serial dilutions of specific dose rifampicin/tobramycin cocktails for each bacterium, and the MIC₅₀ of this new cocktail was determined and compared to the MIC₅₀ of the antibiotics cocktail without silver ion. Result shows that silver ions significantly reduced MIC50 of tobramycin/rifampicin cocktail by 6.5-21 fold. The amount of antimicrobial agent required for inhibition is shown in Table 2, and antibacterial activities of cocktail with silver is shown in Table 3.

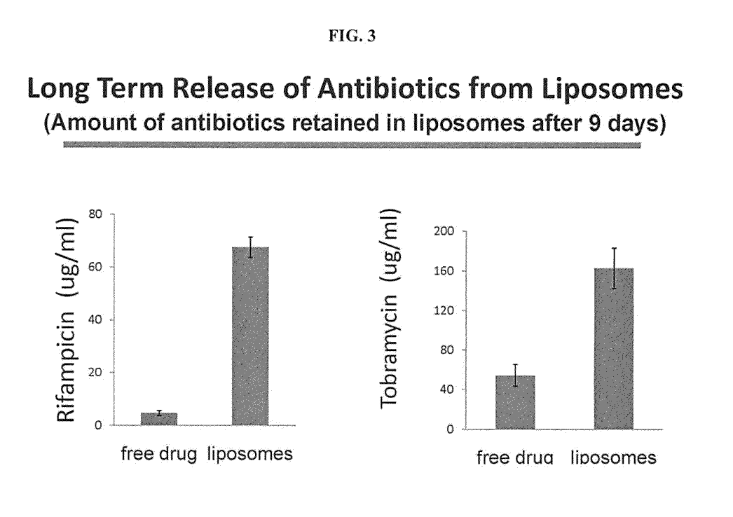

The effect of storage time on antibiotic functionality of tobramycin and rifampicin against A. baumannii were also tested. Tobramycin and rifampicin solutions (1 mg/ml in water and DMSO, respectively) were kept at room temperature (24° C.) or 37° C. for up to 35 days, and the antimicrobial activities of the antibiotics at different time points were tested by applying the antibiotics to A. Baumannii. MIC₅₀ were determined and compared. Results show no significantly change from day 0 to day 35 when stored at 37° C. P values for tobramycin and rifampicin (from day 0 to day 35) are 0.593 and 0.442 respectively (FIG. 3)

TABLE 2
Amount of Antimicrobial Agent required for Inhibition - mg/ml

| Antibiotics | A. bumannii | P. aeruginosa | P. mirabilis | S. aureus |
|---|---|---|---|---|
| Imipenem | 10.25 ± 2.33 | 0.91 ± 0.56 | 1.02 ± 0.01 | 0.02 ± 0.02 |
| Tobramycin | 1.05 ± 0.02 | 0.44 ± 0.12 | 7.2 ± 0.73 | 0.43 ± 0.1 |
| Clindamycin | 2.72 ± 0.68 | >1000 | 23.88 ± 8.43 | 0.004 ± 0.002 |
| Vancomycin | 79.4 ± 4.1 | >1000 | >1000 | 0.82 ± 0.57 |
| Rifampicin | 0.085 ± 0.01 | 19.08 ± 6.14 | 3.77 ± 0.92 | 0.004 ± 0.002 |
| Silver Ion | 1.52 ± 0.73 | 5.34 ± 3.07 | 4.56 ± 1.49 | 13.94 ± 14.21 |

TABLE 3
Antibacterial activities of cocktail with silver

| | | | P. aerualnosa | | P. mirabilis | | S. aureus | |
|---|---|---|---|---|---|---|---|---|
| | A. baumannii | | Tobramycin | Rifampicin | Tobramycin | Rifampicin | Tobramycin | Rifampicin |
| Silver | | | | | | | | |
| Ion | | | | | | | | |
| Mic 50 | 10.4 | 9 | 10.4 | 10.4 | 6.5 | 6.3 | 21 | 21 |
| Mic 25 | 4.2 | 3.6 | 4.1 | 4.1 | 2.5 | 2.4 | 9.3 | 9.3 |
| Mic 12.5 | 2 | 1.73 | 1.6 | 1.6 | 0.74 | 0.74 | 1.13 | 1 |
| Control | 1 | 1 | 1 | 1 | 1 | 1 | 1 | 1 |

### Example 2

### Efficacy of Liposome Encapsulated Antibiotics against Straphylococcus

### Preparation of Liposomes

A previously published method for making liposomes was modified to encapsulate antibiotics, rifampicin and tobramycin (Mugabe C, 2006; Halwani M, 2007). Briefly, a 50 μmol of PPC and 25 μmol of cholesterol were dissolved in 1 ml of chloroform. The solution was dried to form a lipid film with a rotary evaporator at 50° C. under controlled vacuum. The lipid film was flashed with nitrogen gas to eliminate traces of chloroform before hydration. In Step 1 (hydrate), the lipid film was hydrated with 2 ml of sucrose/distilled water (1:1, w/w). The lipid suspension was vortexed for 2 minutes to form multilamellar vesicles, and then sonicated for 10 minutes in an ultrasonic bath (model 2510, Branson). The resulting mixtures were centrifuged at low speed (400 g, 10 min at 4° C.) to remove large vesicles. In step 2 (dehydration-rehydration), the suspension of small unilamellar vesicles was mixed with 1 ml (2.5-40 mg/ml) of antibiotic. Tobramycin was dissolved in dH₂0 and rifampicin dissolved in acetone, respectively. The mixture was then lyophilized overnight. For rehydration, 200 μl of distilled water was added, and the solution vortexed, and incubated for 30 min at 50° C. This step was repeated with 200 μl of PBS (pH 7.2). After incubation, 1.6 ml of PBS was added. The mixture was vortexed and incubated for another 30 min at 50° C. Excess unencapsulated drug was removed by washing with PBS three times (18300 g for 15 min at 4° C.). The encapsulation rate was quantified using an agar diffusion microbiological assay after lipid vesicles were lysed with 0.2% Triton X-100. Triton X-100 did not show inhibitory activity. The mean diameter of liposomes was determined using a 90 Plus Size Analyzer (Brookhaven Instruments Corporation) and Transmission Emission Microscopy (TEM).

### Encapsulation Efficiency of Antibiotic Liposomes

Encapsulation efficiency of the liposomes was determined as the percentage of antibiotics incorporated into vesicles relative to total amount of drug in solution and was calculated using the following equation:

Encapsulation efficiency=C_{vesicles}/C_{vesicles}+Cₛₒₗ)

Where C_{vesicles} is the concentration of the antibiotic entrapped in vesicles (nanoparticles) and Cₛₒₗ is the total concentration of antibiotic in solution.

In Vitro Release of Drugs from Liposomes

One ml of liposome loaded with 1 mg antibiotics (tobramycin or rifampicin) was placed in dialysis tubing and dialyzed over 100 ml of PBS buffer at 37° C. with stirring. Free antibiotic solutions were used as controls. The 100 μl of PBS solution was taken at 0, 2, 4, 6, 12, 24, 48, 72, 96, 128, 156, 180, 204, 228 hours. The released antibiotics were quantified using an agar diffusion microbiological assay.

### Quantification of Entrapped Antibiotics

Concentration of encapsulated antibiotics was determined using an agar diffusion assay using laboratory strains of Staphylococcus aureus (S.a) 12600. Briefly, bacterial suspensions were prepared in Trypticase soy broth (TSB). Bacterial density was adjusted to 0.2 at OD₆₂₀ₙₘ, and the bacterial solution was added into warm (50° C.) Muller Hinton agar (2×10⁷ organisms/ml). The bacterial agar was then poured into a sterile Petri dish and left to solidify for 1 hour at room temperature. Wells of 5 mm diameter were made with a well puncher and filled with 25 μl of sample or standard solutions. The plates were incubated for 18 hour at 37° C. The inhibition zones were measured and the average of duplicate measures was used in data analysis. A standard curve was constructed with known concentrations of free antibiotics (Rifampicin, 0.156-10 μg/ml; Tobramycin, 1.56-100 μg/ml) and was used to estimate concentrations of the entrapped antibiotics that were released from the liposomes. The minimum detection limit of the assay for rifampicin and tobramycin were 0.015 and 1.5 μg/ml, respectively.

The samples were loaded into dialysis tubing and dialyzed over 100 ml of PBS buffer at 37° C. The remaining antibiotics inside the dialysis tubing were determined after 9 days of dialysis.

### Determination of the Minimum Inhibitory Concentration of Antibiotics

To determine the effective concentration of antibiotics to prevent to treat infect, the minimum inhibitory concentration of the antibiotics must first be identified. Free antibiotics in solution and antibiotic-loaded liposomes were serially diluted and inoculated onto agar plates with the bacteria of interest: S. aureus Methicillin-resistant strain (MRSA) BAA-1720 (S.a-R), Acinetobacter baumannii (a.b), BAA-1605, Pseudomans aeruginosa (P.a) 10145, and Proteus mirabilis 4630 (American Type Culture Collection Rockville, Md.). Detailed method is described under quantification of entrapped antibiotics.

### Statistics

All experiments were repeated at least three times. The data were analyzed by ANOVA and Paired Student's t-test to determine whether the differences between two groups were significant.

### Results

Table 4 shows that the average particle sizes of liposomes were approximately 300-500 nm and 200-300 nm for rifampicin and tobramycin, respectively. The average size and encapsulation efficiency varied depending on the amount of antibiotic agent used for liposome formation and the type of antibiotic encapsulated. A decrease in amount of antibiotic agent used for loading reduced both encapsulation efficiency and particle size. There is also a direct relationship between particle size and encapsulation efficiency, and this may explain why Tobramycin-loaded liposomes, have lower encapsulation efficiency. These are smaller particles.

TABLE 4
Particle size and encapsulation efficiency of antibiotic-loaded liposomes.

| | Concentration | Encapsultion | Particle size |
|---|---|---|---|
| Antibiotics | (mg/ml) | Efficiency (%) | (nm) |
| Rifampicin | 20 | 64.8 ± 17 | 543.5 ± 26.1 |
| | 10 | 49.6 ± 1.9 | 533.5 ± 23.3 |
| | 5 | 39.2 ± 9.7 | 497.0 ± 5.7 |
| | 2.5 | 36.9 ± 5.9 | 351.5 ± 21.9 |
| Tobramycin | 20 | 26.7 ± 4.7 | 334.5 ± 65.8 |
| | 10 | 24.3 ± 5.3 | 311.5 ± 91.2 |
| | 5 | 22.5 ± 3.5 | 262.0 ± 73.5 |
| | 2.5 | 19.4 ± 3.7 | 209.0 ± 14.1 |

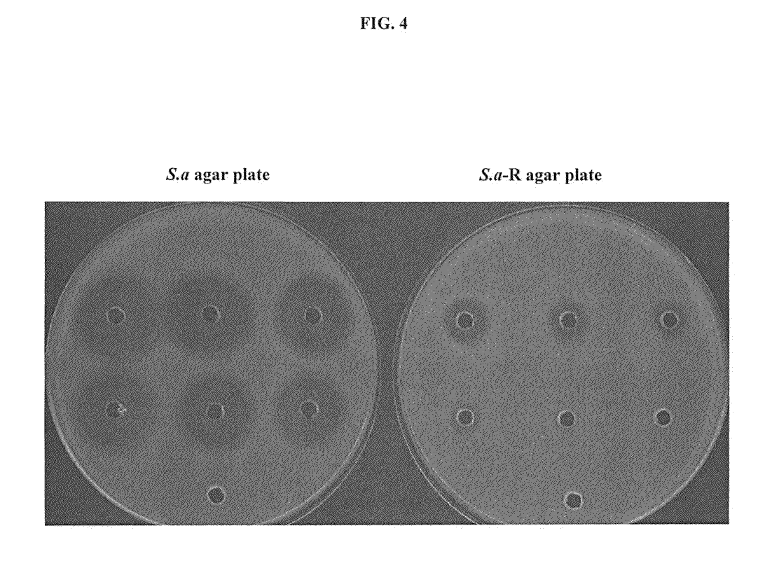

The amounts of antibiotics released over the 7-9 day period were sufficient for inhibiting bacterial growth (data not shown). At day 9, released rifampicin and tobramycin both demonstrated antibacterial activity against S. aureus. The amount of antibiotics retained in liposomes in dialysis tubing compared to free antibiotics is shown in FIG. 4. Results also show that there were no significant differences in the level of inhibition against S.a or MRSA by free rifampicin or rifampicin released from liposomes (data not shown). However, tobramycin-loaded liposomes (right plate, well 1, 2, 3) were much more effective against Sa-R (well 4, 5, 6) (FIG. 4).

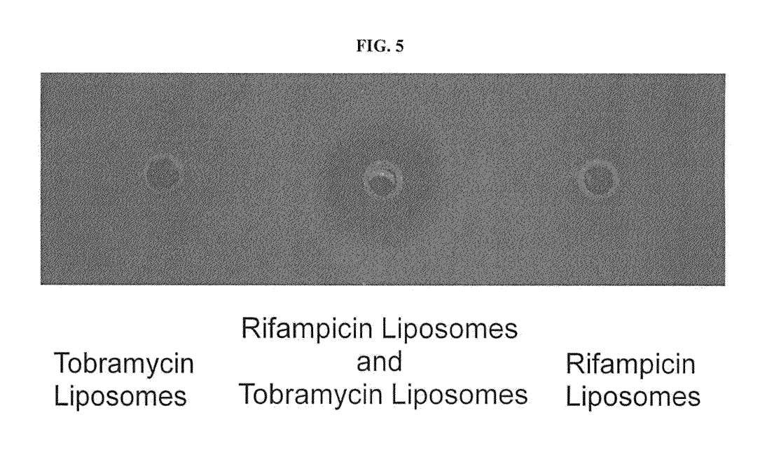

In conclusion, the cocktail with tobromycin and rifampicin was able to reduce the total concentration of antibiotics required to archive bacterial inhibition by as much as up to 70% compared to using single antibiotic. The addition of silver ions into the cocktail was able to further reduce required antibiotics by up to 21 folds. Rifampicin liposomes and Tobramycin liposomes cocktail has enhanced S.a inhibition activities compared to using single antibiotic liposomes (FIG. 5).

### Example 3

### Application of Using Liposome Encapsulated Antibiotics on Implant for Treatment or Prevention of Infection

Coating Implant with Nanoencapsulated Antibiotics

### Liposome Preparation

A 50 μmol of PPC and 25 μmol of cholesterol were dissolved in 1 ml of chloroform in 125 ml round-bottomed flask and dried to a lipid film with a rotary evaporator at 50° C. under controlled vacuum. The lipid film was flashed with nitrogen gas to eliminate traces of chloroform. Rehydration with 2 ml of distilled water/sucrose (1:1, w/w, sucrose to lipid). Sucrose was used to stabilize the liposomes during freeze drying. The lipid suspension was vortexed for 2 min to form multilamellar vesicles and sonicated for 10 minutes in an ultrasonic bath (model 2510, Branson). The resulting mixtures were centrifuged at low speed (400×g, 10 min at 4° C.) to remove large vesicles. The suspension of small unilamellar vesicles was then mixed with 1 ml (5-40 mg/ml) of the target antibiotic. The mixture was then lyophilized overnight (Freeze Dryer, Labconco Corp., Kansas City, Mo.). 200 μl of distilled water was added, and then vortexed, and incubated for 30 min at 50° C. This step is repeated with 200 μl of phosphate-buffered saline (PBS, pH 7.2). After incubation period, 1.6 ml of PBS was added and the mixture was vortezed and incubated for another 30 ml at 50° C.

### Purification of Liposomes:

Excess unencapsulated drug was removed following three rounds of PBS wash (18300 g for 15 min at 4° C.). The pellet was resuspented in 2 ml of PBS.

Coating Liposomes with Chitosan

Dilution of 1% (w/v) chitosan to 0.6% (w/v) by adding of 0.5M sodium acetic. Mixing the liposomes suspensions with an equal volume of 0.6% chitosan. String the mixture for 1 h at room temperature to stabilization and then stored at 4° C. until using. Titanium (Ti6A14v, 5 mm×5 mm square piece) implant and PMMA implants were placed in Acetone for 30 min and in 2% liquid-Nox detergent for 1 hour. It is then rinsed with DI water. Passivity in 35% Nitric acid for 1 hour and rinsed with DI water. The implant is allowed to dry in clean laminar flow hood for 24 hours.

Coating on Titanium Implant with Liposome-Chitosan Complex

Apply 20 ul of liposome-chitosan complex on surface of titanium implant, and make sure the complex cover all surfaces of implants. Air-dry for 24 h in clean flow hood

### Antibacterial Activity of Implant Coated Nanoparticle-Encapsulated Antibiotics

S. aureus was cultured on TSB-agar plate for 18 hours. Make S. aureus suspension in broth, adjust OD600 to 0.2. Add 250 ul of S. aureus in 12.5 ml agar broth (allow to cool down temperature to 50° C.), mixing well, and immediately pour in 10 cm Petri dish. Formalize for 1 hour at room temperature. Place coated titanium implant on the surface of S.a-agar plate Make sure coated surface face down on agar surface. Keep at room temperature for 2 hours and transfer to 37° C. incubators for 18 hours. Measure inhibition ring and make record by taking pictures. Carefully transfer each titanium implant to new S.a-agar precast plate. Repeat the procedure for PMMA implants.

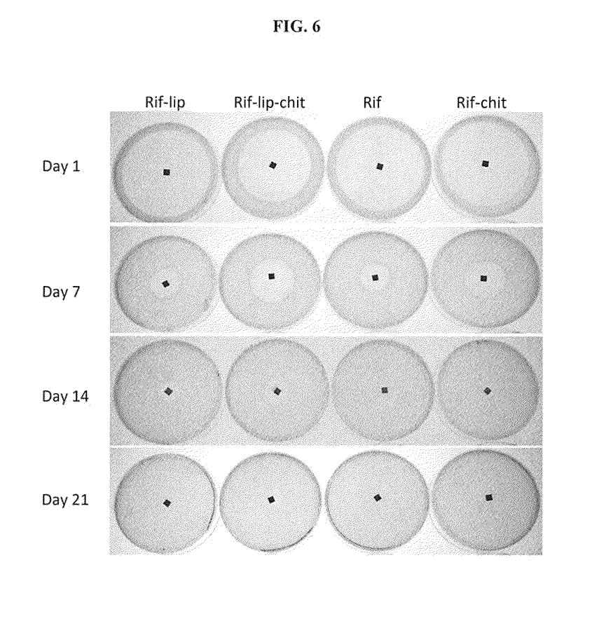

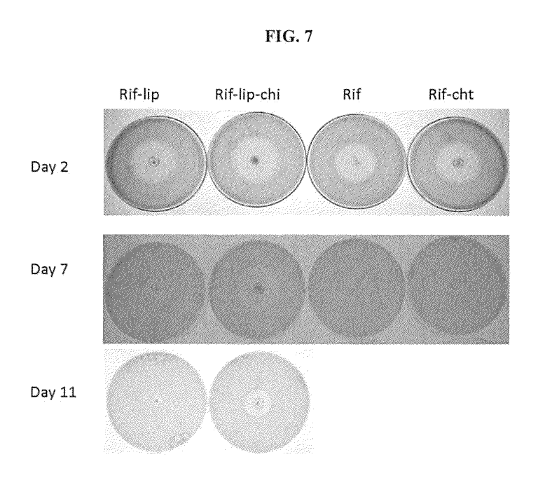

Results for coated titanium implant were shown in FIG. 6 and Results for coated PMMA implant were shown in FIG. 7. Rifampicin encapsulated Liposome coating prolonged the antibacterial effect of the antibiotics. Chitosan stabilized Rifampicin Liposomes and is shown to increase and prolong the antibacterial efficacy of the Rifampicin Liposomes.

### Example 4

### Polymeric Nanoparticles

### Effect of PVA Concentrations on the Formation of Rifampicin Loading PLGA Nanoparticles

PVA serves as a stabilizer in emulsification and NP formation. Therefore, the effect of PVA concentration on the NP sizes and encapsulation efficiency in loading RIF was studied to determine suitable conditions for NP formation. O/W emulsification procedure was to test the effect of PVA concentrations on the formation of PLGA nanoparticles loading rifampicin. Briefly, 2 mg drug and 20 mg PLGA were dissolved in 2.5 ml acetone at room temperature. The resulting solution was slowly dropped into 20 ml H₂O containing different concentrations of PVA (0.5-5%) with vigorous vortexing. The suspension was stirred at approximately 1200 rpm for 4 hrs at room temperature to remove acetone with some water by evaporation. The final volume of the aqueous suspension was collected and then centrifuged at 16,000 rpm, 15° C., for 1 hour (centrifuge, Beckman). NPs were collected and washed (three times) with distilled water containing 0.1% PVA using centrifugation method as described previously. The final pellets (NPs) were suspended and lyophilized by means of Christ Alpha 1-4 lyophilizer (Christ, Osterode, Germany). Particle sizes and encapsulation rates were determined as described in the following relative sections.

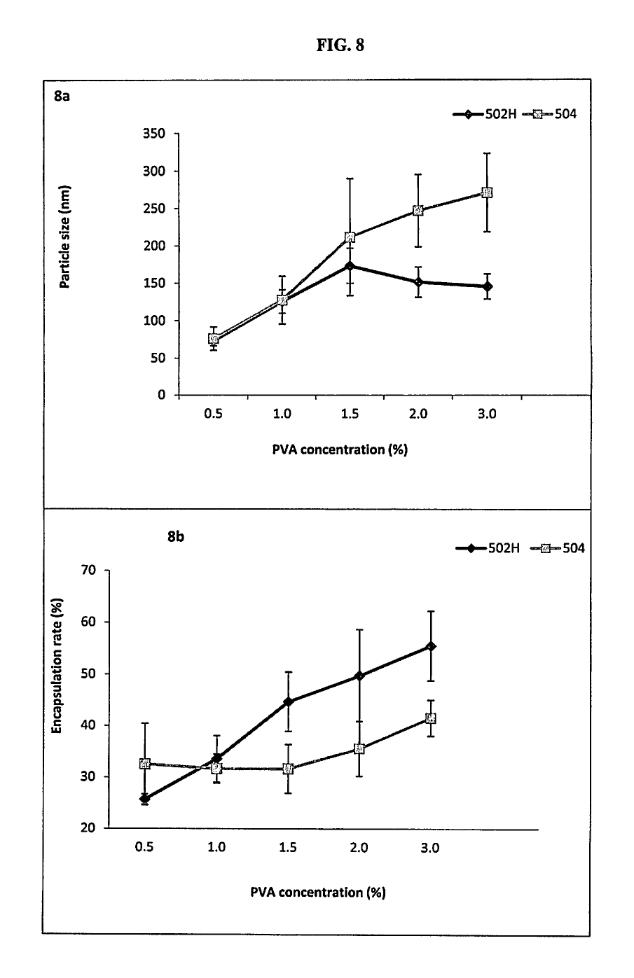

As shown in FIG. 8a, higher concentrations of PVA (>1.5%) resulted in the formation of smaller NPs using PLGA 504 as the polymer to load RIF (P<0.01). A similar trend was seen in PGLA 502H. However, it is interesting to note that very small NPs were able to form under very low PVA concentration (0.5%) despite of different polymer sizes and compositions. This allowed us to design NPs loading RIF with a broad range of sizes including the smaller NPs (<80 nm). In contrast, higher concentrations of PVA (≧1.5%) appeared to correlate with higher encapsulation efficiency when PLGA 504 was used as the polymer (P<0.01) as seen in FIG. 8b. Again, PLGA 502H demonstrated a similar trend, but was not significant (P>0.05).

Influence of Water Phase Volume on the NP Sizes from PLGA as Polymer

W/O/W emulsification procedure was used to prepare nanoparticles to load hydrophilic tobramycin (Tb). In this section, the influence of water phase volume on the NP size formation was determined. PLGA 502H and 504 polymers were used. One milligram Tb was dissolved in different volumes of H₂O (0.125−5 ml). Twenty milligrams PLGA (502H or 504) were dissolved in 2.5 ml acetone. The different concentrations of Tb water solutions were then emulsified individually in the oil phase containing either PLGA 504 or 502H polymers in acetone. The resulting emulsion was slowly dropped into 20 ml of 0.5% PVA under high speed stirring at room temperature for 4 hrs to remove acetone. The final NP suspension was centrifuged at 18,000 rpm, 15° C., for 1 hour. The other treatments including washing and drying nanoparticles are the same as above. NP sizes were measured using size analyzer described below.

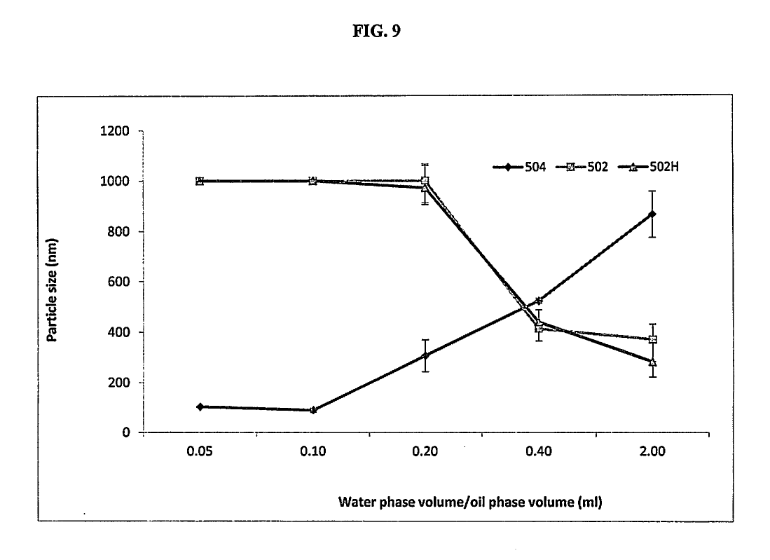

As demonstrated in FIG. 9, water phase volume (0.125−5 ml) altered NP sizes differently when different PLGA polymers were used although oil phase volume remains constant (2.5 ml). Lower water phase volumes (≦0.25 ml) resulted in smaller NPs (<100 nm) when PLGA 504 (MW 45,000-72,000) was used as the polymer. However, when PLGA 502H (MW=7,000-15,000) was used, NP size was increased by approximately 16 folds with lower water phase volumes (from 0.125−0.50 ml to >0.50 ml). This result allowed us to design smaller NPs (<90 nm) for loading hydrophilic drugs using low water phase volumes (≦0.25 ml) from PLGA 504 as polymer.

To understand whether the PLGA 502H COOH terminal group has contributed to the difference in water phase volume effect on NP sizes between 502H and 504, the same experiment demonstrated by FIG. 9 was performed using PLGA 502 which lacks the COOH terminal group. No significant difference was found between PLGA 502 and 502H in either the effect of water phase volume on the sizes of Tb loading NPs (FIG. 9) or that of PVA concentration on the sizes of RIF loading NPs (data not shown).

### Encapsulation Efficiency and Particle Sizes of NPs Loading Antibiotics in Selected Conditions

To characterize encapsulation efficiency and particle sizes of the NPs, different PLGA polymers (PLGA 502H, 503H, 504 and 507 with numbers denoting molecular sizes and letter H denoting terminal group COOH), different experimental conditions, antibiotics (RIF and Tb) were used in the experiments. For loading RIF, 20 mg of each of the 4 different PLGA polymers and 2 mg RIF were dissolved in 2.5 ml of acetone. The solution was then dropped into 20 ml of 1.5% PVA water solution and then stirred to remove acetone; Tb-NPs were prepared by emulsifying 1 ml Tb water solution (2 mg) in 5 ml acetone and then mixed with 20 ml, 0.5% PVA under stirring for 4 hours. NPs in the suspension were harvested and washed by centrifugation. Encapsulation efficiency and particle sizes of NPs loading antibiotics were determined as in relative sections below.

Distribution of Anti-Bacterial Activity Loaded in NPs from Fractions by Differentiation Centrifugations

RIF-NPs were prepared using 20 mg PLGA (either PLGA 502H or 502) and 1 mg RIF in 2.5 ml acetone. The drug and polymer solution was dropped into 20 ml of 0.5% PVA H₂O solution. The detailed methods were described and the NP sizes and encapsulation capacity were analyzed. The suspension of NPs underwent differentiation centrifugations at 12,000 rpm (12 k) for 2 hrs and the supernatant was further centrifuged in ultracentrifuge at 80,000 rpm (80K) at 4° C. for 2 hrs. Anti-S. aureus activities of the pellets (NPs) from 12 k and 80 k and final supernatant from 80 k were determined. The encapsulation capacity (%) was calculated relative to the total antibacterial activity (100%).

### Particle Size Analysis

The mean diameter of nanoparticles and polydispersity index were determined by 90 Plus Size Analyzer (Brookhaven Instruments Corporation). The size distribution analysis was performed at a scattering angle of 90 degrees at room temperature (24° C.) using appropriate dilution of each sample using pure water.

Analysis for Encapsulation Rate of Antibiotics Loaded into Nanoparticles

Encapsulated and unencapsulated antibiotics were analyzed by agar diffusion assay using laboratory strains of S. aureus as indicator organism as described in previous section. Briefly, the bacterial (2×10⁷ bacteria) agar plate was prepared. Wells of 5 mm diameter were made with a well puncher later to be filled with 25 μl of samples or standard solutions. The plate was incubated for 18 hrs at 37° C. The bacterial inhibitory ring was measured in triplicates and the average was used for data analysis. A standard curve was constructed with known concentrations of free antibiotics and utilized to calculate the concentrations of the entrapped antibiotics released from the NPs by 1% acetone. This concentration of acetone did not show any inhibitory activity on the plants. Encapsulation efficiency was determined as the percentage of antibiotic incorporated into the nanoparticles relative to total amount of drug in solution. Encapsulation rate was calculated using the equation below: Encapsulation efficiency (%)=C_{vesicles}/(C_{vesicles}+Cₛₒₗ), where C_{vesicles} is the concentration of the antibiotics entrapped in vesicles and Cₛₒₗ is the concentration of antibiotics unentrapped in vesicles.

### Influence of PLGA Type on Encapsulation Efficiency and Nanoparticle Sizes Loading Rifampicin and Tobramycin

To investigate the in vitro release of loaded drugs from NPs, available methods described were used to prepare NPs loaded with lipohilic rifampicin and hydrophilic tobramycin. PLGA 502H, 503H, 504 and 507 were used as polymers to form NPs to load RIF and Tb in 0/W and W/O/W emulsion, respectively. Nanoparticle sizes and entrapment efficiencies were determined for each PLGA type as shown in Table 5. For NPs loading RIF, NP size was positively correlated with PLGA size. Smaller PLGA 502H and 503H produced the smaller NPs (average 142 nm and 162 nm, respectively) while larger PLGA 504 and 507 formed larger NPs (average 191 and 226 nm, respectively). In contrast, size of NPs loading Tb was negatively correlated with PLGA size. Smaller PLGA 502H and 503H formed larger NPs (average 972 nm and >2,000 nm, respectively) while larger PLGA resulted in smaller NPs (average 354 nm and 560 nm, respectively).

TABLE 5
Encapsulation efficiency and particle sizes of PLGA
nanoparticles loading antibiotics

| | | Encapsulation | |
|---|---|---|---|
| | | Efficiency | Nanoparticle |
| Antibiotics | PLGA type | (%) | Sizes (nm) |
| Rifampicin: | 502H | 59.8 ± 17.3 | 142.3 ± 26.1 |
| | 503H | 47.6 ± 1.9 | 147.6 ± 1.9 |
| | 504 | 51.5 ± 9.7 | 191.6 ± 5.7 |
| | 507 | 41.8 ± 5.9 | 226.9 ± 21.9 |
| Tobramycin: | 502H | 42.2 ± 4.7 | 972.0 ± 65.8 |
| | 503H | 24.3 ± 5.3 | 1,100 ± 91.2 |
| | 504 | 32.5 ± 3.5 | 354.5 ± 73.5 |
| | 507 | 19.6 ± 1.5 | 560.5 ± 14.1 |

### Nanoparticle Morphology

Transmission electronic microscopy (TEM) was used to image the morphology of the nanoparticles. A drop of nanoparticle suspension containing 0.01% of phosphotungstic acid was placed on a carbon film coated on a copper grid for TEM. Observation was fulfilled at 80 kV in microscopy.

The morphology of NPs prepared from PLGA 504 in 3% PVA were characterized by transmission electron microscopy (TEM). The RIF-loaded nanoparticles showed a spherical and regular morphology with the particles obtained by this preparation technique 2.2. In addition, TEM images confirmed their homogeneous particle size distribution, as already suggested by measurements shown in Table 6.

TABLE 6
Nanoparticle sizes and distribution of Anti-bacterial activity
from fractions after differentiation centrifugations

| | | Distribution of anti-bacterial activity | | | |
|---|---|---|---|---|---|
| | | (encapsulation capacity) (%) in | | | |
| | | different fractions of centrafugation | | | |
| | | | 23K x g | 80K x g | Final |
| PLGA | NP sizes (nm) | Total | pellets | pellets | Supernatant |
| 502 | 66.25 ± 9.8 | 100.0 | 23.0 ± 2.2 | 16.9 ± 3.9 | 60.1 ± 4.1 |
| 502H | 62.1 ± 13.4 | 100.0 | 37.2 ± 10.4 | 12.7 ± 3.3 | 50.1 ± 17 |

In Vitro Release of Rifampicin and Tobramycin from Loaded Nanoparticles

The in vitro drug release from loaded PLGA nanoparticles was performed by the methods described with some modification. Briefly, 0.6 ml of drug-loaded nanoparticles was suspended in 20 mM phosphate buffered normal saline (PBS), pH7.4 in an Eppendoff tube flatting on a rack at 37° C. For each cycle, the NP suspensions were centrifuged at 14,500 rpm for 20 min. The supernatant was collected and stored at −20° C. The precipitated NPs were re-suspended in an equal volume of PBS and placed at 37° C. The cycle was repeated and supernatants were collected at day 1, 3, 5, 7, 9, 11, 14, 17, 21, 24, and 28. All samples including supernatants and final NPs re-suspension were analyzed as above. The analysis of drug release from NPs was performed by quantitative analysis of antibacterial activity.

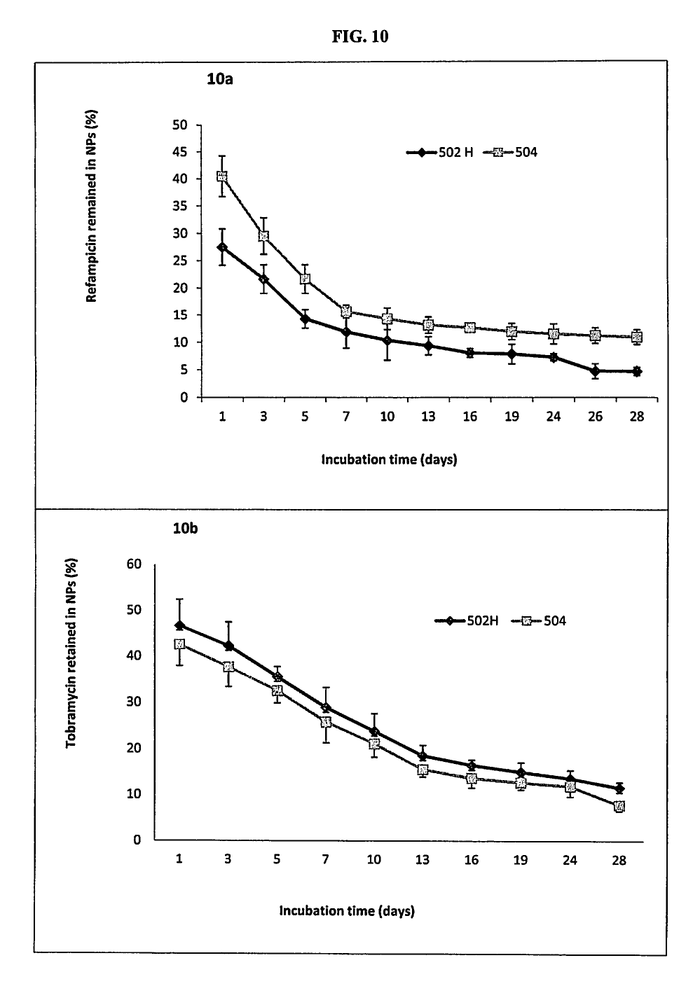

Average NP sizes and encapsulation capacity were showed in Table 5. Antibiotic in vitro release studies of NPs were performed over 28 day period in phosphate-buffered saline (PBS) at 37° C. FIG. 10 suggested that the anti-bacterial activities from both of RIF (FIG. 10a) and Tb (FIG. 10b) were maintained over this period. After incubation of the NPs for 28 days, both of RIF-NPs and Tb-NPs retained 5-10% anti-bacterial activities against S. aureus (FIG. 10). Interestingly, the larger NPs either loading RIF or Tb released the loaded drugs more slowly than those smaller NPs did (FIGS. 9a and 9b).

### Antimicrobial Capability of Antibiotic Loaded NPs on Multiple Bacterial Strains

To obtain better antibacterial activities, the small NPs (average <80 nm) were prepared from PLGA 504 and 502H polymers. For loading lipophilic RIF, the small NPs were prepared following the methods described. However, for smaller Tb-NPs preparation, 1 mg Tb was dissolved in 0.25 ml H₂O and the solution was emulsified in 2.5 ml acetone containing 20 mg of PLGA polymer. The resulting emulsion was immediately suspended in 20 ml 0.5% PVA by high speed stirring for 4 hrs. The final suspension including all nanoparticles and unloaded drug were used in the experiments. Bacterial strains, A. baumannii, P. aeruginosa, P. Miris, S. aureus and S. aureus methicillin resistant strain were used for assessment of the antimicrobial capabilities of antibiotic loaded NPs against multiple bacterial strains. The 50% minimum inhibitory concentration needed to form inhibition ring (MIC_{fir}) was determined by filling serially diluted free antibiotics in solution and antibiotic-loaded NPs in the wells of the bacterial agar plates with the selected strains (S.a, MRSA, A.b, P.a, and P.m). Further details were described above in method section “Determination for encapsulation rate of antibiotics loaded into nanoparticles”. Antibacterial activities of the free drugs were used as control. The MIC_{fir} was calculated based on the standard curve of quantitative analysis from free drug.

Preliminary results showed smaller NPs have stronger anti S. aureus activity. However, we were not able to recover all the small size NPs from the NP suspension. Therefore, methods to prepare smaller NPs either carrying RIF or Tb were developed using PLGA 504 as polymer. Whole antibiotic-NP suspensions (average particle sizes 75 nm for RIF-NPs and 80 nm for Tb-NPs) were used in the observation. To understand the capacities of NP loaded antibiotics against the five selected bacterial strains, MIC_{fir} on the agar plate were measured for both free antibiotics and NP loaded antibiotics against each bacterial strain. As seen in Table 6, either RIF-NPs or Tb-NPs were able to increase the anti-bacterial activity against all five strains (A.b, S.a, P.a, P.m, MRSA and Kp) by 4-12 times.

TABLE 3
The capabilites of RIF-NPs on anti-multiple infectious bacterial strains
differentiation centrifugations

| | 50% minimum inhibitory concentrations (MIC₅₀) needed | | | | | |
|---|---|---|---|---|---|---|
| | for growth ring formation (μg/ml) | | | | | |
| PLGA | A.b | P.a | P.m | S.a | MRSA | K.p |
| Free RIF | 45 ± 2.6 | 185 ± 15.8 | 26 ± 3.5 | 0.02 ± 0.001 | 0.02 ± 0.001 | 45.0 ± 3.6 |
| NPs- | 5.0 ± 1.5 | 6.0 ± 0.5 | 4.5 ± 0.7 | 0.01 ± 0.001 | 0.005 ± 0.00 | 18.5 ± 2.2 |
| RIF | | | | | | |
| Free | 7.1 ± 0.5 | 5.6 ± 0.2 | 3.1 ± 0.2 | 2.2 ± 0.16 | >200 | 0.38 ± 0.05 |
| TOB | | | | | | |
| NPs- | 1.0 ± 0.09 | 1.3 ± 0.1 | 0.9 ± 0.1 | 0.3 ± 0.01 | 50 ± 10.5 | 0.09 ± 0.01 |
| TOB | | | | | | |

Aa = A. baumannii,
P.a = aeruginosa,
Pm = P. Aeruginosa,
Sa = S. Aureus,
K.p = K. pneumoniae and
MRSA = S. aureus methicillin resistant strain.
N = 3.

In summary, results showed that sufficient drug concentrations were released to exert antibacterial activities against S. aureus. Moreover, approximately 10.5% of the drug activity remained in the NPs at the end of 4 weeks. The antibacterial results suggested that NPs increased antibiotic activity against Staphylococcus aureus (ATCC 12600), Acinetobacter baumannii (BAA-1605), Pseudomonas aeruginosa 4-8 times.

The results also suggested that the changes of first water phase volume (0.125-5 ml) vs constant oil phase volume (2.5 ml acetone) and the second water phase volume (20 ml 0.5% PVA) showed significant differences in the formation of NP sizes. Results in RIF-NPs preparation showed small polymer, PLGA 502H (Mw7-15 k) and 5031-1 (Mw 14-23K) and 502 (7-15 k) produced smaller NPs (150, 180, and 140 nm), while large molecules of PLGA 504 (30-60 k) and 756 (60-90 k) formed larger RIF-NPs with mean diameters of 190 and 250 nm. However, the formation of Tb-NPs did not follow this pattern. In this case, polymers with smaller molecular weights (PLGA 502, 502H and 503) resulted in the formation of larger Tb-NPs in the same conditions compared with those with larger molecular weights.

In term of the effect of NPs on bacterial infections associated with implant and wound multiple infections. The results suggested smaller NPs showed better antibacterial activity compared with the larger NPs either extracting RIF or Tb. Smaller NPs (<90 nm) resulted in the significant increase of antibacterial activity against five bacterial strains that are frequently involved in wound and implant infections approximately 4-8 times when RIF-NPs or Tb-NPs activities were compared with respective free drug activity.

In vitro release data shows the initial release was dominated for all formulations during first three days of the release, being greater than approximately 50%. The early release could be from the diffusion release of the drugs distributed at or just beneath the surface of the NPs. Subsequent release may be mainly due to the diffusion of drug molecules through the polymeric matrix of the NPs. In addition, the results suggested that drug loaded in the larger NPs (RIF-NPs from PLGA 504 and Tb-NPs from 502H) released the drug more slowly than those in the smaller NPs (RIF-NPs from PLGA 502H and Tb-NPs from 504).

Results showed that RIF-NPs possessed remarkable anti-S. aureus activity including wild type and its resistant strain (MIC=0.0005 μg/ml). However, they only showed weak antibacterial activity against the other strains studied in our experiments. On the contrary, Tb-NPs had weaker anti-S. aureus activity and was ineffective against MRSA due to resistance. However, they had high antibacterial activity against the other stains studied when compared with RIF-NPs. This suggest different NPs loading with various antibiotics may be used in combination to form a pharmaceutical formulation against a broad range of bacteria.

### Prophetic Example 5

### Biocompatibility Testing

If the nanoparticle of the invention is to be implanted or otherwise applied or administered in the body of a subject, the material should be biocompatible. To assess biocompatibility, cells (e.g., a fibroblast, keratinocytes or neurons cell line) can be seeded onto the nanoparticles impregnated on to an implant or coated in a culture dish. If the fibroblasts are able to replicate and attach to the composition, the composition is likely to be biocompatible. Alternatively, the composition can be implanted into the body of a subject (e.g., a mouse, rat, dog, pig, or monkey) for a specified time, and then removed to evaluate the number and/or health of the cells attached to the composition. The ability of the implant to support growth of fibroblasts is particularly important when infiltration of cells and deposition of an extracellular matrix on the composition are desired in vivo.

### Prophetic Example 6

### Efficacy of the Implants Impregnated with Antibiotic Encapsulated Nanoparticles

For in vivo testing, implant impregnated with antibiotic encapsulated nanoparticles can be implanted into an animal (e.g., a mouse, rat, dog, pig, monkey, or rabbit). Localized infection is created by using Acinetobacter baumannii and the animal is monitored for signs of, pain, redness, discharge, swelling, or heat at the site of a wound or intravenous line and fever. These observations and length of signs of infection are then compared to those of animals with only PMMA implant, and animals with only PMMA implant but given oral antibiotic treatment.

While preferred embodiments of the present invention have been shown and described herein, it will be obvious to those skilled in the art that such embodiments are provided by way of example only. Numerous variations, changes and substitutions will now occur to those skilled in the art without departing from the invention. Accordingly, it is intended that the invention be limited only by the spirit and scope of the appended claims.

### REFERENCES

1. Arap W, Kolonin M G, Trepel M, Landenranta J, Cardo-Vila M, Giordano R J, Mintz P J, Ardelt P U, Yao V J, Vidal C I, Chen L, Flamm A, Valtanen H, Weavind L M, Hicks M E, Pollock R E, Botz G H, Bucana C D, Koivunen E, Cahill D, Troncoso P, Baggerly K A, Pentz R D, Do K A, Logothetis C J, Pasqualini R. “Steps toward mapping the human vasculature by phage display.” Nat. Med. 2002 February; 8(2):121-7.
2. Arkadiusz K, Gubernator J, Przeworska E, and Stasiuk M. “Liposomal drug delivery, a novel approach” Acta Biochim. Polo., 2000, 47, 639-649.
3. Flint P W, Li Z B, Lehar M, Saito K, Pai S I. “Laryngeal muscle surface receptors identified using random phage library.” Laryngoscope. 2005 November; 115(11): 1930-7.
4. Fukuda M N. “Screening of peptide-displaying phage libraries to identify short peptides mimicking carbohydrates.” Methods Enzymol. 2006; 416:51-60
5. Giorgio G, Marina M, Casazza R, Bonin M, Bassetti M, Cruciana M, Antimicrobial Agent and Chemotherapy, 1998, 42, 3014-17.
6. Henry C M. “Cover Story; Drug Delivery” C&E News Washington, 2002, 80, 39-47
7. Johanson C E, Duncan J A, Stopa E G, Baird A. “Enhanced prospects for drug delivery and brain targeting by the choroid plexus-CSF route.” Pharm Res. 2005 July; 22(7):1011-37. Epub 2005 Jul. 22.
8. Kolonin M G, Sun J, Do K A, Vidai C I, Arap W. “Synchronous selection of homing peptides for multiple tissues by in vivo phage display” The FASEB Journal, 2006, 20, 99-107.
9. Mugabe C, Haleani M, Azghzni A O, Lafrenie R M, Omri A. “mechanism of Enhanced Activity of Liposome-Entrapped Aminoglycosides against Resistant Strains of Pseudomonas aeruginosa.” Antimicrobial Agents and Chemother., 2006a, 50, 2016-2022.
10. Mugabe C, Azghani A O, Omri A. “Preparation and Characterization of Dehydration-rehydration vesicles loaded with aminiglycosides and microlide antibiotics.” Int. J. Pharm., 2006b, 307, 244-250.
11. Muller-Goymann C C. “Physiochemical characterization of colloidal drug delivery system such as reverse micelles, vesicles, liquid crystals and nanoparticles for topical administration” European Journal of Pharmaceutics and Biopharmaceutics, 2004, 58, 343-356
12. Nandi I, Bari M, Joshi H. “Study of isopropyl myristate microemulsion system containing cyclodextrins to improve the solubility of 2 model hydrophobic drugs”, 2003, 4, 1-9
13. O'Connor K H, Banga J P, Darmanin C, El-Kabbani O, Mackay I R, Rowley. “Characterization of an autoreactive conformational epitope on GAD65 recognized by the human monoclonal antibody b78 using a combination of phage display, in vitro mutagenesis and molecular modeling.” J. Autoimmun. 2006 May; 26(3):172-81. Epub 2006 Mar. 24.
14. O'Connor K H, Königs C, Rowley M J, Pustowka A, Dietrich U, Mackay I R. “Requirement of multiple phage displayed peptide libraries for optimal mapping of a conformational antibody epitope on CCR5.” J Immunol Methods. 2005 April; 299(1-2):21-35. Epub 2005 Feb. 1.
15. Pridgen E M, Langer, R, Farokhzad, O D., Nanomed. 2007, 2(5), 669-80.
16. Torchilin V P. “Structure and design of polymeric surfactant-based drug delivery system” Journal of Control Release, 2001, 73, 137-172
17. Valadon P, Garnett J D, Testa J E, Bauerle M, Oh P, Schnitzer J E. “Screening phage display libraries for organ-specific vascular immunotargeting in vivo.” Proc Natl Acad Sci USA. 2006 Jan. 10; 103(2):407-12. Epub 2005 Dec. 29.
18. Whaley S R, English D S, Hu E L, Barbara P F, Belcher A M. “Selection of peptides with semiconductor binding specificity for directed nanocrystal assembly.” Nature. 2000 Jun. 8; 405(6787):665-8.
19. Xu Y, Ramsland P A, Davies J M, Scealy M, Nandakumar K S, Holmdahl R, Rowley M J. “Two monoclonal antibodies to precisely the same epitope of type II collagen select non-crossreactive phage clones by phage display: implications for autoimmunity and molecular mimicry.” Mol. Immunol. 2004 June; 41(4):411-9.
20. Yael V R, Gunther J, Terman G W, Chavkin C. “Mu opioid analgesia and analgesic tolerance in two different mouse strains” Proc. West. Pharmacol. Sol., 2000, 43, 15-17.
21. Boger A, Heini P, Windolf M, Schneider E., Eur. Spine J, 2007, Aug. 23.
22. Frankel B M<D'Agostino S, Wang C., J. Neurosurg. Spine, 2007, 7, 47-53
23. Nandi I, Bari M, Joshi H. AAPS PharmSciTech., 2003, 4, 1-9.
24. Torchilin V P., Journal of Control Release, 2001, 73, 137-172.
25. Alipour M, Suntres Z E, Lafrenie R M, Omri A. J. Antimicrob Chemother. 65:684-93.2010
26. Lanio M E, Luzardo M D, Alvarez C, Martinez Y, Calderon L, Alonso M E, Zadi B, Gregoriadis G, Craig D Q, Disalva A. J Liposome Res. 18:1-19. 2008.
27. Mugabe C, Azghani A O, Omri A. Int J. Pharm. 307: 244-50. 2006.
28. Halwani M, Mugabe C, Azghani A O, Lafrenie R M, Kumar A, Omri A. J Antimicrob Chemother. 60: 760-9. 2007.

What is claimed is:

1. A pharmaceutical formulation comprising a plurality of nanoparticles, said nanoparticles encapsulating a therapeutically effective amount of one or more therapeutic agents, and an application of the formulation to an implant before surgery provide for extended release of said therapeutic agents.

2. The pharmaceutical formulation of claim 1, wherein said nanoparticles are selected from the group consisting of micelle, inverse micelle, unilamellar liposome, multilamellar liposome, polymeric nanoparticle and a combination thereof.

3. The pharmaceutical formulation of claim 1, wherein said nanoparticles are selected based on the therapeutic agents prescribed for treatment.

4. The pharmaceutical formulation of claim 1, wherein said therapeutic agent is an antibiotic, an antibacterial compound, an growth hormone, an pain medication, or an anti-cancer drug.

5. The pharmaceutical formulation of claim 4, wherein said antibiotic is selected from the group consisting of Imipenem, rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

6. The pharmaceutical formulation of claim 1, wherein said nanoparticles are coated onto an implant.

7. The pharmaceutical formulation of claim 6, wherein said implant is a joint implant, a cranial implant, a hip implant or a bone implant.

8. The pharmaceutical formulation of claim 7, wherein said implant is a PMMA implant, hydroxyapatite implant, hydrogel or titanium implant.

9. The pharmaceutical formulation of claim 6, wherein said nanoparticles are coated on the surface of said implant using a physiological acceptable coating material to stabilize said nanoparticles.

10. The pharmaceutical formulation of claim 9, wherein said coating material is a modified PMMA compound or Chitosan.

11. The pharmaceutical formulation of claim 6, where said nanoparticles are coated on the surface of an implant using phage display.

12. A method for providing extended release of antibiotic agent to a target site comprising:
a) producing a plurality of nanoparticles, said nanoparticles encapsulating a therapeutically effective amount of one or more therapeutic agents; and
b) administering said nanoparticles to said target site;
wherein said therapeutic agents is release over a extended period of time.

13. The method of claim 12, wherein said nanoparticle is selected from the group consisting of micelle, inverse micelle, unilamellar liposome, multilamellar liposome, polymeric nanoparticles and a combination thereof.

14. The method of claim 12, wherein said therapeutic agent is an antibiotic, an antibacterial compound, an pain medication, a growth hormone, a anti-cancer drug.

15. The pharmaceutical formulation of claim 4, wherein said antibiotic is selected from the group consisting of imipenem, rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

16. The method of claim 12, wherein said implant is a joint implant, a cranial implant, a hip implant or bones implant.

17. The method of claim 12, wherein said implant is a PMMA implant, hydroxyapatite implant, hydrogel or titanium implant.

18. The method of 12, wherein the concentration and type of nanoparticles and the antibiotic agents are selected based on a prescribed treatment regimen.

19. The method of claim 12, further comprising:
a) coating said nanoparticles onto an implant; and
b) implanting said implant in a patient.

20. The method of claim 19, wherein said nanoparticles are coated on the surface of said implant using a physiological acceptable coating material to stabilize said nanoparticles.

21. The method of claim 20, wherein said coating material is a modified PMMA compound or Chitosan.

22. The method of claim 19, wherein the physiologically acceptable coating material comprises a first component selected from the group consisting of polycaprolactone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

23. The method of claim 19, wherein the physiologically acceptable coating material comprises polycaprolactone as a first component admixed with nitrocellulose as a second component.

24. The method of claim 19, where said nanoparticles are displayed on the surface of an implant using phage display.
